(19) United States
(12) Patent Application Publication
(10) Pub. No.: US 2026/0355405 A1
(43) Pub. Date: Nov. 12, 2026

(54) INCISION AND DRAINAGE DEVICE

(71) Applicant: Bromley Bio Med LLC, Horseheads, NY (US)

(72) Inventor: Alexis Bromley, Horseheads, NY (US)

(73) Assignee: Bromley Bio Med LLC, Horseheads, FL (US)

(21) Appl. No.: 19/468,977

(22) Filed: Feb. 3, 2026

Related U.S. Application Data
(60) Provisional application No. 63/760,772, filed on Feb. 20, 2025.

(51) Int. Cl. A61M 1/00

(52) U.S. Cl. CPC A61M 1/84; A61M 1/60; A61M 1/76; A61M 2250/00

(57) ABSTRACT

Representative embodiments of an incision and drainage device are described herein. The incision and drainage device includes a body having a syringe mount, a septum defining a blade opening and a drainage opening, a foot defining a cavity, and an actuator lobe. An actuator advances a blade enclosed within the actuator lobe through the blade opening to form an incision and retracts the blade to a shielded position. With the foot contacting skin, a syringe or other vacuum source coupled to the mount is actuatable to aspirate wound fluid through the drainage opening into the vacuum source.

### CROSS REFERENCE TO RELATED APPLICATIONS

This application claims priority to U.S. provisional patent application number 63/760,772 filed on Feb. 20, 2025, entitled “Incision and Drainage Device,” the disclosures of which are incorporated herein by reference in their entirety.

### BACKGROUND

### Technical Field

The disclosures herein relate generally to medical devices and methods for performing incision and drainage procedures, including accessing and draining subcutaneous fluid collections such as abscesses, cysts, and other fluid or semi-fluid material. More particularly, the disclosures relates to devices that may be positioned at a skin surface to facilitate formation of an incision and removal of a fluid collection from a subcutaneous region using a negative-pressure source such as a syringe or other vacuum reservoir.

### State of the Art

Subcutaneous fluid collections requiring drainage are extremely common. Examples of such collections include cysts, abscesses, post-traumatic collections including seromas and hematomas, and the like. Many resolve spontaneously and may therefore managed expectantly. Frequently, however, patients present with fluid collections under the skin which may be infected, enlarging, painful, or having other indications for drainage. By some estimates, infected subcutaneous fluid collections (abscesses) needing drainage are responsible for about two percent (2%) of the greater than 140 million emergency room visits annually in the United States—nearly 3 million visits for infected subcutaneous fluid collections requiring drainage.

Drainage may be achieved in the physician's office, clinic, or hospital emergency room through aspiration with a needle coupled to a syringe, or incision by a blade, typically a scalpel. Incision is required for infected collections to create an open, persistent skin opening to allow for continued drainage of infected fluid which the body may continue to produce until the infection is resolved. Though typically a very minor procedure, incision and drainage (“I&D”) must be performed by personnel skilled in the procedure to (1) achieve complete or near-complete removal of the fluid; and (2) avoid unnecessary pain or discomfort to the patient. A scalpel is used to incise the skin and create an opening through which the fluid may freely drain. Although surgeons and many emergency room physicians are experienced and skilled at incising superficial, subcutaneous fluid collections, many physicians and other clinicians are not.

Most commonly, clinicians use a scalpel blade to perform I&D procedures. For inexperienced and uncomfortable clinicians, this is problematic. Tentative movements to incise the skin inadequate to effectively drain the collection may be performed. Often, infected fluid collections are aspirated with a needle and re-accumulate because the skin has not been incised creating an opening to allow for ongoing drainage. Failure to remove all (or almost all) of the material from a cyst, abscess, or other localized fluid collection leads to prolonged healing, infection, further tissue damage, and recurrence. Massaging the area around a drainage incision is an imprecise way to remove liquid material that may remain. Moreover, incising a fluid collection is messy, with spillage of infections fluid on skin and other surfaces proximate to the incision site, and with aerosolization of infections pathogens. Furthermore, because incision and drainage is commonly performed using a separate cutting instrument (e.g., a scalpel blade) to puncture or incise the treatment site, followed by manual expression of fluid and/or aspiration using a separate syringe or suction device, I&D requires multiple separate equipment items. Alternatively, in some circumstances, the clinician may massage the surrounding subcutaneous area to encourage drainage, and gauze or other packing material may be placed in the site to collect remaining fluid over a period of time after the procedure. Such workflows may involve multiple instruments and handling steps, and may be time consuming and messy, with potential exposure to infections (biohazard) material and increased sharps-handling events.

Accordingly, there is a need for improved incision and drainage devices and methods that may streamline a procedure by integrating incision and aspiration functionality in a single device, and providing controlled blade advancement and retraction and enabling collection of aspirated material in a receptacle. In some embodiments, such devices may facilitate more efficient evacuation of fluid from a subcutaneous fluid collection and may reduce handling of exposed sharps and drained material during and after the procedure. Such devices may also decrease hesitancy when a clinician with limited experience must perform an incision and drainage procedure.

### SUMMARY

The following presents a simplified summary to provide a basic understanding of certain aspects of the subject matter disclosed herein. This summary is not comprehensive nor is it intended to identify key/critical elements of the claimed invention, or to delineate the scope thereof. Rather, its purpose is to present some example concepts in a simplified form as a prelude to the more detailed descriptions presented later. It is presented in view of the aforementioned needs and example shortcomings set forth herein above.

Disclosed is an incision and drainage device comprising a body having a syringe mount, a septum comprising a blade opening and a drainage opening, a foot extending from the septum bounded by a rim and defining a foot cavity, and an actuator lobe; a vacuum source coupled to the syringe mount; an actuator coupled to the actuator lobe; and a blade mechanically coupled to the actuator interface, wherein the actuator interface is configured to advance and retract the blade; wherein advancement and retraction of the blade in response to the actuator interface establishes a fluid connection between the foot cavity and a subcutaneous fluid collection in a human or an animal, whereupon a subcutaneous fluid collection may be aspirated through the drainage opening into an aspiration volume fluidly coupled to the syringe mount.

In some embodiments, the body further comprises an elongated syringe receiver. In some embodiments, the vacuum source is a syringe removably mounted to the syringe mount. In some embodiments, the vacuum source comprises a Vacutainer. In some embodiments, the incision and drainage device further comprises a closing mechanism configured to contain aspirated material after aspiration, the closing mechanism comprising two semi-circular members configured to close around a diameter of the Vacutainer.

In some embodiments, the blade is manually advanced and is spring-loaded to retract. In some embodiments, the septum comprises a long axis and the body comprises a long axis, wherein the body long axis is orthogonal to the septum long axis. In some embodiments, the septum comprises a long axis and the body comprises a long axis, wherein the body long axis is nonorthogonal to the septum long axis. In some embodiments, the body long axis is about fifteen degrees nonorthogonal to the septum long axis. In some embodiments, the body comprises a grip feature. In some embodiments, the rim is disposed opposite the septum and configured for contacting the skin of an animal, including a human.

In some embodiments, the actuator lobe comprises a slot oriented parallel to a long axis of the body; the actuator comprises a blade mount coupled to an actuator interface by a tab, wherein movement of the tab is constrained by the slot, the tab is positioned with the blade mount inside the actuator lobe in a retracted position prior to an actuation sequence, the actuator interface is positioned outside the actuator lobe of the body, and the tab is disposed through the slot, wherein the blade mount is configured for movement through the actuator lobe parallel to a long axis of the body in response to movement of the actuator interface transmitted through the tab. In some embodiments, the device further comprises a detent in the actuator lobe configured to retain the tab in a retracted position wherein the blade does not traverse the blade opening of the septum.

Disclosed is an incision and drainage device comprising a body having a syringe mount, a septum comprising a blade opening and a drainage opening, a foot coupled to the septum, and an actuator lobe; a syringe coupled to the syringe mount; an actuator coupled to the actuator lobe; and a blade mechanically coupled to the actuator, wherein the actuator is configured to advance and retract the blade; wherein advancement and retraction of the blade in response to the actuator establishes a fluid connection between the foot cavity and a subcutaneous fluid collection in an animal, such as a human, whereupon a wound fluid may be aspirated through the drainage opening into a syringe coupled to the syringe receiver.

Disclosed is a method of use for an incision and drainage device to remove fluid from a subcutaneous fluid collection comprising step coupling a selected vacuum source to an incision and drainage device having a tissue puncture means and an actuator mechanism for the tissue puncture means; positioning a foot of the incision and drainage device at a skin site overlying a subcutaneous fluid collection, wherein the foot of the incision and drainage device contacts a skin surface at the skin site; actuating the tissue puncture means to cause entry followed by removal of the tissue puncture means from the subcutaneous fluid collection; actuating the vacuum source causing removal of fluid from the subcutaneous fluid collection into the vacuum source and removing the foot of the incision and drainage device from the skin surface.

In some embodiments, the method further comprises a step exchanging the vacuum source. In some embodiments, the vacuum source comprises a Vacutainer. In some embodiments, the vacuum source comprises a syringe, and wherein actuating the vacuum source comprises withdrawing a plunger of the syringe.

In some embodiments, the method further comprises maintaining aspirated material within the vacuum source after removing the incision and drainage device from the skin site. In some embodiments, the blade is retained in an actuator lobe following blade removal from the subcutaneous fluid collection during actuation of the vacuum source.

Disclosed is an incision and drainage device comprising a body having a syringe mount, a septum comprising a blade opening and a drainage opening, a foot coupled to the septum, and an actuator lobe. A syringe may be coupled to the syringe mount, an actuator may be coupled to the actuator lobe, and a blade may be mechanically coupled to the actuator. The actuator may be configured to advance and retract the blade such that, in response to advancement and retraction, a fluid connection is established between a cavity of the foot and a subcutaneous fluid collection in an animal, whereupon wound fluid may be aspirated through the incision and from the foot through the drainage opening into the syringe.

In some embodiments, the body further comprises an elongated syringe receiver, wherein the syringe may be positioned within the elongated syringe receiver to be removably mounted to the syringe mount. In some embodiments, the septum comprises a long axis and the body comprises a long axis, wherein these long axes may be orthogonal or nonorthogonal, including about fifteen degrees nonorthogonal. In some embodiments, the body comprises a grip feature. In some embodiments, the body comprises a perimeter wall coupled to the septum and having a rim disposed opposite the septum, the rim being configured for contacting a skin surface.

In some embodiments, the actuator lobe comprises a slot oriented parallel to the long axis of the body. The actuator may comprise a slide coupled to a button by a tab, with the slide positioned inside the actuator lobe, the button positioned outside the body, and the tab passing through the slot, such that the slide is configured for movement through the actuator lobe parallel to the long axis of the body in response to movement of the button transmitted through the tab. In some embodiments, the tab further comprises a guide positioned within a detent disposed on the actuator lobe and configured to move within the detent in response to movement of the tab along the slot. In some embodiments, the actuator lobe includes a detent configured to retain the tab in a retracted position in which the blade does not traverse the blade opening of the septum.

Disclosed is a method of use for an incision and drainage device to remove fluid from a subcutaneous fluid collection comprising steps identifying a skin site proximate to the subcutaneous fluid collection; contacting the skin site with a rim of a foot of an incision and drainage device having a blade and a syringe; actuating the blade to cause blade entry followed by blade removal from the subcutaneous fluid collection; actuating the syringe causing removal of fluid from the subcutaneous fluid collection into the syringe; and removing the incision and drainage device. In some embodiments, the method further comprises replacing the syringe and/or locking the blade in a retracted position. In some embodiments, at least one actuating step is automated.

In some embodiments, a vacuum source for aspiration may comprise a syringe or a Vacutainer, and the device may be configured to interface with the particular vacuum sources and/or of a particular size, such as syringe sizes from 5 cubic centimeters (“cc”) to 100 cc., for example. In some embodiments, the device includes a closing mechanism configured to contain aspirated material after an incision and drainage procedure is completed, such as a closing mechanism comprising two flexible semi-circular pieces configured to close around a diameter of a Vacutainer. In some embodiments, the blade is manually advanced from a protective casing mounted in an actuator lobe and spring-loaded to retract, and a blade retractor mechanism may be disposed on slide and actuated by a retractor button coupled to a spring.

Elements and features from any of these above-mentioned and other embodiments may be used in combination with one another in accordance with the general principles described herein. These and other embodiments, elements, features, and advantages will be more fully understood upon reading the following detailed description in conjunction with the accompanying drawings and claims.

### BRIEF DESCRIPTION OF THE DRAWINGS

The present invention is illustrated by way of example—not by way of limitation—in the several figures of the accompanying drawings and wherein like reference numerals refer to similar elements.

FIG. 1 is a perspective view of an example embodiment of an incision and drainage device coupled to a syringe and including an actuator configured to translate along a long axis of a body.

FIG. 2 is an anatomic cutaway view of an embodiment of an incision and drainage device positioned at a skin surface and actuated to access a subcutaneous fluid collection for aspiration via an attached syringe.

FIG. 3 is a perspective view of a body of an embodiment of an incision and drainage device showing a grip feature and a longitudinal slot configured to guide an actuator tab.

FIG. 4A is a perspective view of an embodiment of an incision and drainage device body in an upright configuration including a grip feature and a longitudinal actuator slot through which an actuator tab is movable.

FIG. 4B is a perspective view of the incision and drainage device having a body of FIG. 4A in a tilted orientation showing a longitudinal slot configured to guide an actuator tab along a body long axis.

FIG. 5A is a perspective transparent view of an embodiment of an incision and drainage device in a generally upright orientation.

FIG. 5B is a perspective transparent view of an alternative embodiment of an incision and drainage device in a tilted orientation.

FIG. 6 is a perspective view of an alternative embodiment of an incision and drainage device including a body supporting a syringe and an actuator configured to advance and retract a blade.

FIG. 7 is a perspective view of an alternative embodiment of a device body lacking an elongated syringe received and including a base configured to contact skin, a grip recess, an actuator lobe with a longitudinal slot, and an upper interface forming a septum configured to couple to a syringe.

FIG. 8A is a perspective view of an actuator coupled to a blade of an incision and drainage device.

FIG. 8B is an alternative perspective view of the actuator coupled to the blade of an incision and drainage device.

FIG. 9 is a partial sectional view of an incision and drainage device illustrating an internal blade actuation assembly and a fluid path between a foot cavity and a syringe interface.

FIG. 10 is an enlarged sectional view through an actuator lobe.

FIG. 11A is a sectional view of the incision and drainage device in a retracted configuration with a blade positioned within an actuator lobe prior to incision.

FIG. 11B is a sectional view of the incision and drainage device in an incising configuration with a blade advanced through a septum and outward from a foot.

FIG. 11C is a sectional view of the incision and drainage device in a post-incision configuration with the blade retracted to a shielded position within the actuator lobe.

FIG. 11D is a sectional view of the incision and drainage device during aspiration with a syringe plunger withdrawn.

FIG. 12 is a flow chart illustrating a method of using an incision and drainage device.

### DETAILED DESCRIPTION

Before the present articles, devices, and methods are disclosed and described, it is to be understood that the aspects of the examples described below are not limited to the specific embodiments set forth. As such, the disclosed articles, devices, and methods may vary within the intended scope of these disclosures. It is also to be understood that the terminology used herein is for the purpose of describing particular aspects only and is not intended to be limiting.

Various representative example embodiments of an incision and drainage device and methods of use are described in detail herein. Many types of fluid collections within or immediately beneath the skin (subcutaneous fluid collections) may be drained using the disclosed devices and methods. The section headings used herein are for organizational purposes only and are not to be construed as limiting the described subject matter.

The devices and methods set forth in these disclosures are not limited to treating cysts and abscesses, rather, the devices and methods may be used to treat other fluid collections requiring incision and drainage.

### Definitions

As used herein, the singular form of a noun includes the plural unless otherwise stated. As used herein, “or” includes “and” unless otherwise stated. As used herein, “including” and alternative forms such as “includes” and “included” is not limiting. Any range disclosed herein will be understood to include the endpoints and all values between the endpoints.

As used herein, “material” broadly refers to the fluid or semi-fluid contents contained within a cyst, an abscess, or the like. The specific composition of the material can vary depending on various factors, such as location and underlying cause. In a cyst, for example, the material is contained within a sack-like structure. This material can be watery, a thick fluid, a suspension, or a semi-solid paste. Cysts and abscesses for example, may contain serous fluid or “pus,” namely serious fluid containing large amounts of cellular debris, inflammatory cells, proteins, lipids, blood, and microorganisms such as bacteria, fungi, or parasites.

As used herein, “user” refers to a clinician qualified by training to incise and drain a subcutaneous fluid collection. Nonexclusive examples include doctors, physician assistants, nurse practitioners, and the like.

As used herein, “proximal” means nearer to the patient or further from a user. “Proximal” refers to a relative position of certain components of an I&D device disposed more proximate to the skin of a patient than other components disposed closer to a user of the I&D device.

As used herein, “distal” means away from the patient, i.e., nearer to the user with respect to an I&D device or its individual components. The terms “distal” and “proximal” are used herein to relate the relative positions of structures with respect to one another using the patient as a common reference point. The structures may include an I&D device or its individual components. For example, “proximal” means a structure or reference point of an I&D device which is disposed closer to the patient (further from the user) relative to a more “distal” structure of the I&D device. The relationship assumes the device is in use, however, the relative positions of the user and the patient are assumed regardless of whether a user or a patient are present.

As used herein, “scalpel” is used generically to mean a medical sharp, i.e., a medical device configured to incise (cut) or otherwise mechanically pierce skin or other tissues. “Scalpel” includes scalpels, other blades or knives, solid or hollow needles, and the like.

As used herein, “animal” means any animal, including a human.

For purposes of reading the description of the various implementations below, the following descriptions of the sections of the Specification and their respective contents are provided.

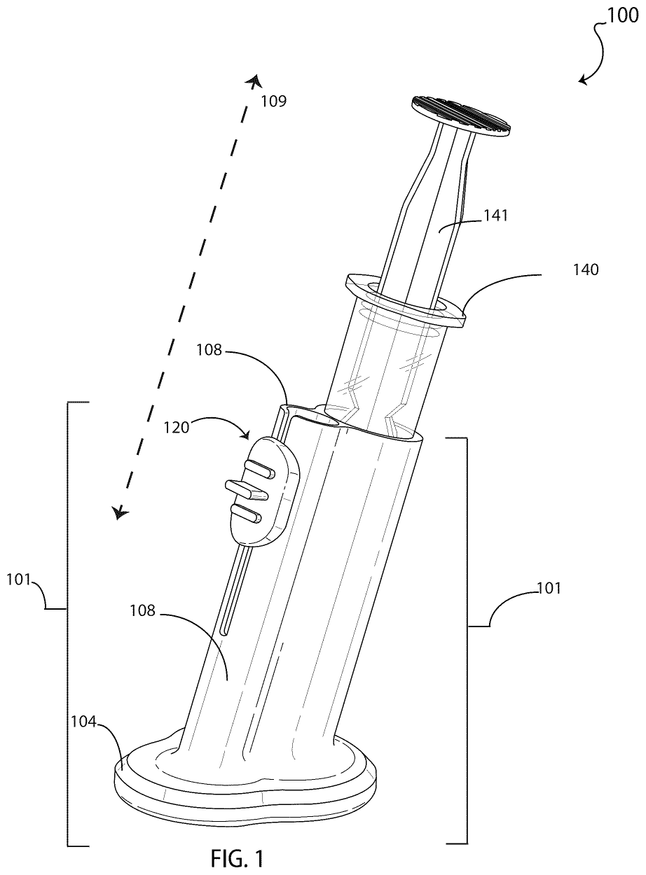

FIG. 1 is a perspective view of an example embodiment of an incision and drainage device coupled to a syringe and including an actuator configured to translate along a long axis of a body. FIG. 1 shows an incision and drainage device 100 assembled with a syringe 140 to form a combined incision and aspiration instrument. The incision and drainage device 100 includes a body 101 that supports the syringe 140 and carries an actuator interface 120 accessible from an exterior of body 101. In the illustrated perspective, syringe 140 is received at an upper region of body 101 such that a longitudinal direction of the assembled system aligns with a body long axis A_{B}.

In some embodiments, body 101 defines an inner volume 107 that provides space for internal components and fluid communication pathways associated with incision and drainage operation. The body long axis A_{B} extends along body 101 and provides a reference direction for placement and movement of components supported by body 101. Additionally, body 101 includes a foot 104 base region that broadens relative to the body 101 upper region to provide a stable interface for manual handling and for positioning the device 100 against a skin surface during use, while maintaining the syringe 140 in a supported orientation relative to the inner volume 107.

In some embodiments, actuator interface 120 is disposed on body 101 and is arranged for user manipulation while the device 100 is held by the user. In some embodiments, actuator interface 120 is positioned on an outer surface of actuator lobe 108 of body 101. Actuator interface 120 is positioned adjacent to body long axis A_{B} and is movable relative to the body 101 in a direction having a component parallel to body long axis A_{B}. Furthermore, actuator interface 120 is operatively coupled to a blade assembly housed within actuator lobe 108 such that user input at actuator interface 120 causes advancement and retraction of a blade 121 relative to a distal end region of body 101, with blade 121 remaining shielded within actuator lobe 108 of body 101 prior to actuation and after retraction.

In some embodiments, syringe 140 functions as a negative-pressure source that is fluidly coupled to inner volume 107 through an interface within the body 101, thereby enabling aspiration of a subcutaneous fluid collection after an incision is formed. Syringe 140 is mounted to the body 101 in a manner that supports axial alignment with the body long axis A_{B}, wherein syringe 140 may be removed and replaced to exchange receptacles while retaining body 101 and actuator interface 120 as a single handheld assembly, in some embodiments. Furthermore, cooperative operation of actuator interface 120 and syringe 140 enables sequential incision formation and subsequent aspiration without transferring device 100 away from the treatment site between steps of an I&D procedure.

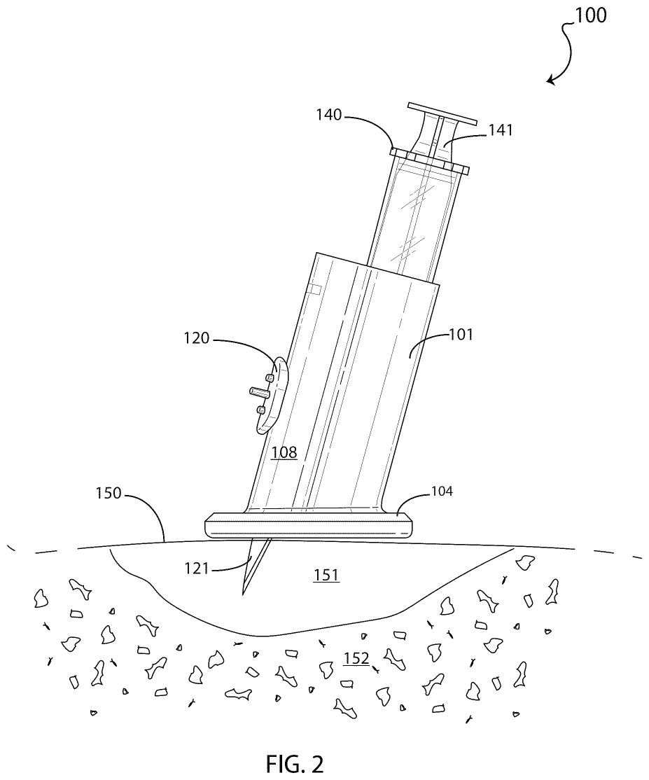

FIG. 2 is an anatomic cutaway view of an embodiment of an incision and drainage device positioned at a skin surface and actuated to access a subcutaneous fluid collection for aspiration via an attached syringe. FIG. 2 shows an anatomic cutaway view in which an incision and drainage device 100 is positioned at a skin surface 150 for access to a subcutaneous fluid collection 151 that has formed within a subcutaneous tissue 152. Device 100 includes a body 101 supporting a syringe 140 and an actuator interface 120 disposed within actuator lobe 108 and projecting onto an exterior of body 101 for user manipulation while device 100 is held against skin surface 150. In the illustrated orientation, a body long axis A_{B} extends along an elongated dimension of body 101 corresponding to a direction along which syringe 140 is carried by body 101.

Body 101 is positioned with a distal region of device 100 contacting the skin surface 150 such that a distal opening of device 100 overlies a target region associated with the fluid collection 151. Additionally, the cutaway anatomy depicts fluid collection 151 as located beneath the skin surface 150 and surrounded by subcutaneous tissue 152 wherein access to fluid collection 151 is provided through an incision formed through the skin surface 150 into the subcutaneous tissue 152. In some embodiments, body 101 supports syringe 140 in a fixed spatial relationship to the distal region of the device 100 during placement at the treatment site.

In some embodiments, actuator interface 120 is operatively coupled to a blade 121 so that movement of the actuator interface 120 advances blade 121 from actuator lobe 108 of body 101 to traverse the skin surface 150 and enter subcutaneous tissue 152. FIG. 2 shows blade 121 extending into fluid collection 151 to create an access path from the skin surface 150 to an interior region of the fluid collection 151. Furthermore, subsequent movement of the actuator interface 120 retracts blade 121 back toward the body 101 to reduce exposure of blade 121 after incision formation while the device 100 remains positioned at the skin surface 150.

In some embodiments, syringe 140 is fluidly coupled through body 101 to foot 104 of device 100 such that withdrawal of a syringe plunger 141 generates negative pressure causing fluid from fluid collection 151 to flow through the incision and into syringe 140. Aspiration occurs while the distal region of the body 101 remains in contact with the skin surface 150, thereby supporting maintenance of a seal between the device 100 and the tissue adjacent the fluid collection 151. Furthermore, the aspirated wound fluid is directed from the fluid collection 151 through an internal flow path of device 100 and into syringe 140 for containment.

In one aspect, body long axis A_{B} provides a reference for coordinated manipulation of actuator interface 120 and syringe 140 during an incision-and-aspiration sequence. In some embodiments, actuator interface 120 is positioned partially within an actuator lobe 108 disposed on body 101 to permit user input while syringe 140 remains supported by body 101 and blade 121 is advanced along a trajectory that intersects skin surface 150 to reach the fluid collection 151 within subcutaneous tissue 152. FIG. 2 also depicts a workflow in which the device 100 creates an incision with blade 121 and aspirates fluid collection 151 via syringe 140 without repositioning body 101 away from skin surface 150 in a separate step.

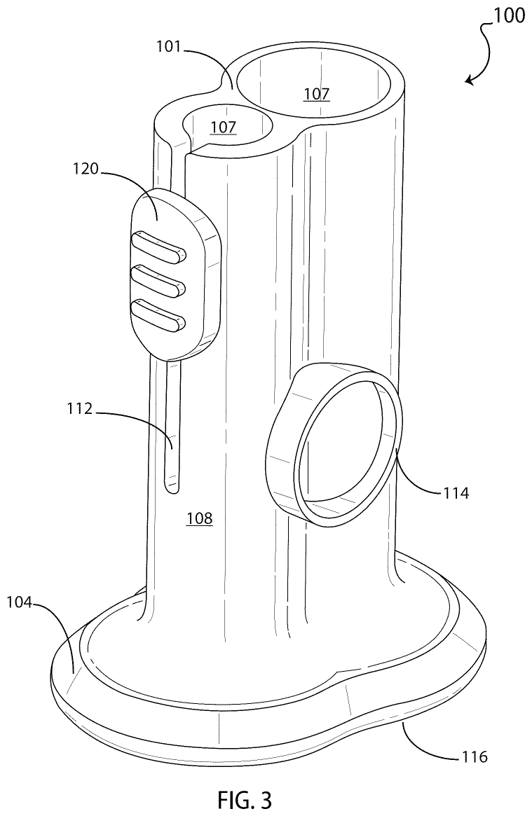

FIG. 3 is a perspective view of a body of an embodiment of an incision and drainage device showing a grip feature 114 and a longitudinal slot 112 configured to guide an actuator tab 123. FIG. 3 shows device 100 wherein body 101 is shown to define external handling features and internal cavities for carrying components described with respect to FIGS. 1-2. Body 101 includes an upper region configured to receive a syringe and a lower region that broadens to form foot 107. Body 101 defines inner volume 107 that extends through body 101 and a fluid communication pathway between a distal drainage opening region and the syringe interface. In some embodiments, inner volume 107 is configured to receive syringe 140. Actuator lobe 108 of body 101 provides space for housing a retracted blade 121 and an actuator interface 120.

In some embodiments, body 101 includes slot 112 disposed along a sidewall and extending in a longitudinal direction parallel with long axis A_{B} of body 101. Slot 112 is configured to constrain movement of a tab 123 during blade actuation, with tab 123 projecting outward through the slot 112 to provide a user-accessible interface. Additionally, tab 123 cooperates with the slot 112 to guide translation of an internal actuator component, such as a slide 122, for example, within actuator lobe 108. Slot 112 provides a mechanical boundary that limits travel of tab 123 to a selected range of motion associated with advancing and retracting blade 121.

In some embodiments, body 101 further comprises grip feature 114 configured to support manual handling by a user positioning device 100 at a treatment site. Grip feature 114 is shown as an opening through a lateral protrusion of the body 101, wherein grip feature 114 is configured to receive a user finger to resist rotation of the body 101 during manipulation of tab 123, in some embodiments. Grip feature 114 may be positioned adjacent a mid-region of body 101 to enable concurrent stabilization of body 101 and translation of tab 123 along the slot 112, such as while urging actuator interface 120 in a direction parallel with body long axis A_{B}. Grip feature 114 cooperates with the broadened distal region where body 101 forms foot 104 to provide multiple contact points for controlling placement of the device 100 against skin surface 150.

FIG. 3 additionally shows a rim 116 extending around an outer perimeter of foot 107. In some embodiments, rim 116 contacts skin surface 150 during use of device 100 to orient, stabilize, and partially seal foot 104 of body 101 against skin surface 150. Rim 114 is formed from the lowest edge of material forming foot 104, in some embodiments. In some embodiments, rim 116 is a separate piece coupled to foot 104 as a gasket interposed between foot 104 of body 101 and skin surface 150 when in use. Note that inner volume 107 is arranged to communicate negative pressure from an attached syringe 140 to foot 107 to draw fluid from fluid collection 151 after a fluid path is established by blade actuation. Positioning foot 104 of body 101 at a skin surface 150 aligns foot 107 over fluid collection 151 while tab 123 is moved along slot 112 to perform an incision-and-retraction sequence. Furthermore, the configuration shown in FIG. 3 enables sequential blade actuation and aspiration while body 101 may remain fixed by the user in a selected orientation relative to the target site.

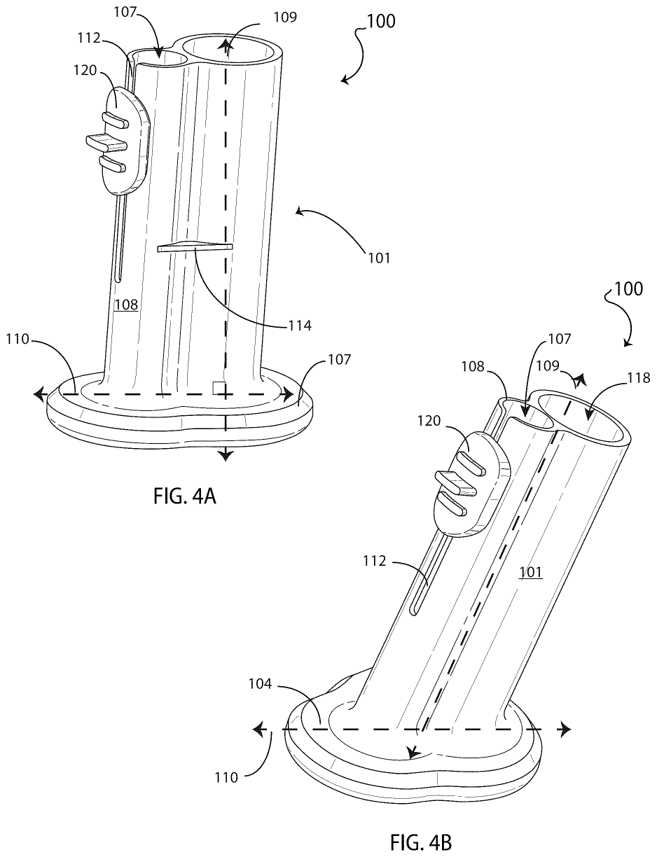

FIG. 4A is a perspective view of an embodiment of an incision and drainage device body in an upright configuration including a grip feature and a longitudinal actuator slot through which an actuator tab is movable. As in some embodiments, FIG. 4A shows incision and drainage device 100 wherein body 101 is in an upright orientation to depict exterior handling and actuation features used during an incision-and-aspiration procedure. Body 101 extends along body long axis A_{B} from a lower base region, i.e., foot 104, toward an upper opening region, i.e. syringe receiver 118, with body long axis A_{B} providing a reference direction for user manipulation actuator interface 120 of a blade actuation assembly described elsewhere in the present disclosure. Additionally, FIG. 4A depicts a lateral side of body 101 whereupon an actuator interface 120 is positioned for access while a user grasps body 101. The upright orientation shown in FIG. 4A corresponds to a placement condition wherein the user may align device 100 relative to a skin site while actuation and aspiration steps are performed without exchanging instruments.

In one aspect, the body 101 defines an inner volume 107 that extends within the body 101 and provides space for internal components and flow passages associated with incision and drainage operation. The inner volume 107 is arranged to accommodate translation of a blade carrier and to provide a fluid communication path between a distal interface region of the device 100 and a proximal interface region, such as a syringe received 118, e.g., configured to couple to a negative-pressure source, including but not limited to syringe 140. Inner volume 107 additionally supports routing of aspirated wound fluid away from a treatment site when a drainage path is established following an incision event. Furthermore, inner volume 107 is positioned within body 101 so that exterior handling forces applied to body 101 by a user can be reacted throughout wall structures surrounding the inner volume 107 while the device 100 remains positioned at the tissue surface.

As present in some embodiments, FIG. 4A shows a slot 112 disposed on an outer wall of actuator lobe 108 of body 101 and extending in a direction aligned with the body long axis A_{B}, wherein slot 112 defines a constrained travel path for a tab 123 that projects outward from an internal actuator component. Tab 123 is coupled to and interposed between an actuator mechanism, such as a slide 122 and actuator interface 120, in some embodiments. Actuator interface 120 is configured to be engaged by a user's finger and translated along the slot 112 to transmit motion into the inner volume 107 via tab 123 for advancing and retracting a blade relative to a distal end of the device 100. Slot 112 additionally cooperates with tab 123 to limit the range of motion available to the tab 123 and to guide the tab 123 along a repeatable trajectory referenced to the body long axis A_{B}. Locating tab 123 extending through slot 112 enables body 101 to serve as a guide surface that stabilizes actuator motion while the device 100 is held against tissue.

In some embodiments, body 101 includes a grip feature 114 positioned on a side of the body 101 to support manual stabilization during actuation of the tab 123 along the slot 112. In some embodiments, grip feature 114 is configured as an opening or recess within body 101 that receives a user finger to provide a reaction surface opposing forces applied to the tab 123, thereby assisting with maintaining a selected device orientation relative to the body long axis A_{B}. Grip feature 114 is located between the upper opening region of body 101 and foot 107 to permit concurrent access to the grip feature 114 and the tab 123 while the device 100 is supported by the user's hand. Furthermore, the spatial relationship between the grip feature 114, the slot 112, and the tab 123 enables coordinated one-hand manipulation in which body 101 may be retained in position as tab 123 is translated to perform an incision-and-retraction sequence prior to aspiration through the inner volume 107. In some embodiments, grip feature 114 is an irregular surface, abrasive surface, sticker, decal, or other high-friction surface feature configured to facilitate non-slip grasping and stabilization of body 101 during operation of device 100.

In some embodiments, including that shown in FIG. 4B incision and drainage device 100 comprises body 101 in a tilted non-orthogonal orientation relative to the orientation depicted in FIG. 4A. In the illustrated perspective, body long axis A_{B} extends along body 101 and is presented as inclined relative to foot 107 of body 101, thereby depicting a body configuration in which a proximal opening region of the body 101 is laterally offset from a distal footprint of device 100. This tilted presentation conveys that body 101 may support a coupled syringe (not shown) along the body long axis A_{B} while positioning a distal interface of device 100 at a treatment site. This nonorthogonal configuration illustrated in FIG. 4B provides visual context for how a user interface represented by actuator interface 120 may remain accessible when the device 100 is held in an angled hand position during use. In some use cases, it is desirable to have body 101 in an angled, non-orthogonal orientation with respect to a long axis AF of foot 107 to facilitate use in an anatomic region were other anatomic structures may preclude easy use of device 100 with body 101 having an orthogonal orientation with respect to long axis A_{F}, such as in the device 100 shown in FIG. 4A.

In some embodiments, actuator lobe 108 of body 101 defines inner volume 107 extending within body 101, with inner volume 107 representing a cavity that may house a blade actuation assembly. In some embodiments, inner volume 107 additionally defines at least a portion of a fluid communication pathway between a distal interface region, such as foot 104, and a proximal syringe interface. The non-orthogonal inclination of body long axis A_{B} shown in FIG. 4B corresponds to an arrangement wherein inner volume 107 is also inclined, such that internal components and flow passages may be routed along an inclined axis while a distal region of the device 100 contacts tissue at a non-included axis. This tilted orientation supports placement of device 100 at skin surface 150 while positioning an attached vacuum source (not shown) at an orientation that can reduce interference with adjacent patient anatomy or clinician hand position.

In some embodiments, FIG. 4B further depicts slot 112 disposed on body 101 and extending in a direction having a component parallel to body long axis A_{B}, with slot 112 defining a constrained travel path for tab 123. Additionally, tab 123 projects outward through slot 112 to provide a user-accessible feature that transmits motion from an external contact surface into the inner volume 107 to drive internal translation of an actuator component (not shown). Furthermore, the placement of slot 112 along an inclined body 101 indicates that tab 123 may be advanced and retracted along a guided trajectory referenced to body long axis A_{B} while the device 100 is maintained at the treatment site, thereby supporting repeatable blade deployment and blade shielding sequences described elsewhere in the present disclosure.

In some embodiments, the cooperative geometry of body 101, slot 112, and tab 123 shown in FIG. 4B provides an actuation interface that remains exposed in the tilted configuration of the device 100. Additionally, translating tab 123 along slot 112 is constrained by wall structures of body 101 adjacent slot 112, which can limit lateral deflection of the tab 123 during actuation and promote controlled movement along body long axis A_{B}. Furthermore, the inclined orientation of body 101 depicted in FIG. 4B supports a workflow in which the device 100 is positioned against tissue while tab 123 is manipulated to form an incision and return a blade to a shielded condition within inner volume 107, after which aspiration through the device 100 may be performed using a coupled syringe or other vacuum source.

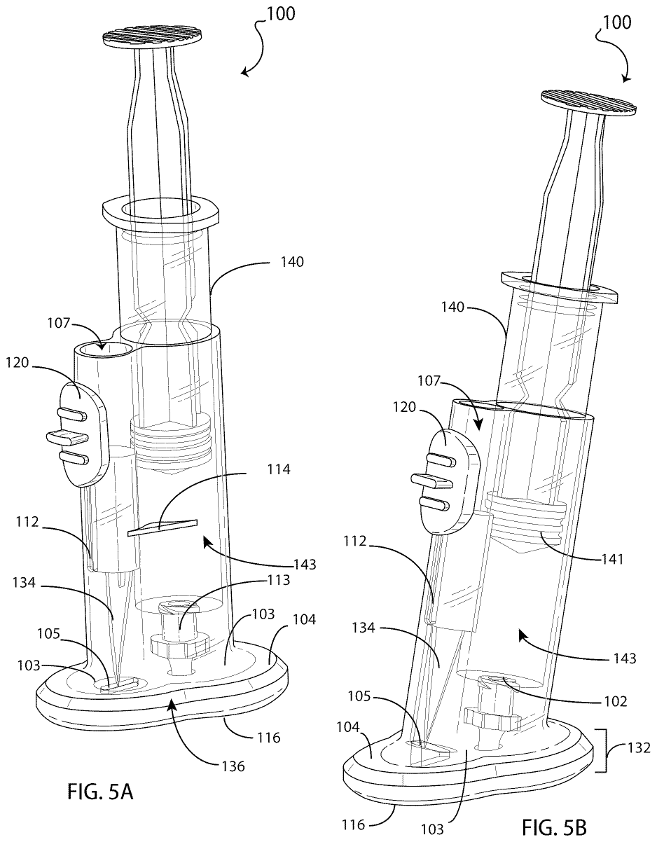

FIG. 5A is a perspective transparent view of an embodiment of an incision and drainage device in a generally upright orientation. FIG. 5A shows device 100 in an upright orientation to depict internal component placement within device 100 during an incision-and-aspiration procedure. The transparent rendering conveys how a syringe or similar aspiration reservoir and a blade actuation assembly are carried within a common housing so that cutting and aspiration functions are performed while the device 100 remains positioned at a treatment site. The upright orientation depicts an arrangement in which a proximal region of device 100 supports a user-manipulated negative-pressure source while a distal region defines a tissue-facing interface. Also, FIG. 5A provides visual context for spatial relationships between internal cavities, actuator-guided translation features, and a distal region through which a tissue puncture means, such as a blade or a needle, can be advanced and retracted.

In some embodiments, device 100 carries syringe 140 in an axial alignment relative to the device housing formed by body 101 such that withdrawal of a syringe plunger 141 generates negative pressure that is communicated into an internal flow path of device 100. The transparent view depicts the syringe barrel seated within an upper syringe receiver 118 of device 100 and shows shaft of plunger 141 extending outward from the barrel for user access. Additionally, the syringe barrel is shown positioned so that an outlet region of the syringe communicates with an interior of device 100 rather than directly to ambient surroundings, thereby supporting aspiration through the device 100 while rim 116 or the distal region of foot 104 is placed against a skin surface The upright orientation of device 100 shown in FIG. 5A depicts a configuration wherein a user can stabilize device 100 at foot 104 while manipulating plunger 141 at the proximal region to aspirate fluid flowing from a subcutaneous collection.

As in some embodiments, FIG. 5A additionally depicts an internal blade path and a user-accessible actuator interface 120 interface positioned on a side of actuator lobe 108 of device 100, with the actuator interface mechanically coupled to a tissue puncture means 134 disposed within the housing. Tissue puncture means 134 is illustrated as a number 11 scalpel blade in FIGS. 5A-B and throughout the several drawing figures, but this is intended to be representative of many possible embodiments of a tissue puncture means as set forth and described further in the paragraphs herein below. The transparent rendering shows a tissue puncture means 134 (here, a blade) extending along a guide region toward a blade opening 105 within septum 103 and positioned so that translation of a slide 122, or alternative internal actuator member, advances blade 121 out of actuator lobe 104 through blade opening 105 for tissue entry and retracts blade 121 back through blade opening 105 for shielding within the housing formed by actuator lobe 108 of body 101. The actuator interface 120 is disposed on an exterior region of device 100 and is coupled to the internal actuator member through an opening in the housing, such as slot 112 in some embodiments, thereby enabling user input to be transmitted into the device 100 while maintaining enclosure of internal components. The upright, orthogonal orientation also supports an understanding that the blade trajectory and the actuator travel direction can be coordinated along body long axis A_{B} of device 100 to provide controlled blade extension and return.

According to an embodiment, FIG. 5A also conveys a fluid communication pathway within the device 100 between a distal tissue-facing region and the syringe carried by the device 100. The transparent view depicts an internal cavity extending from foot 104 of device 100 toward the syringe interface so that, after an incision is formed and the blade is returned to a shielded position, the negative pressure produced by the syringe draws wound fluid through the distal region and into the syringe. Additionally, the distal region is shown as foot 104 having a widened base structure that provides a contact perimeter for placement on skin and for supporting a seal condition adjacent the incision site during aspiration. Furthermore, the internal pathway illustrated in FIG. 5A supports routing of aspirated material away from the tissue-facing region and toward the syringe volume for containment during and after aspiration.

In one aspect, the transparent view of FIG. 5A depicts device 100 as a single handheld assembly that maintains the spatial relationship between the distal interface, the internal blade actuation assembly, and the proximal aspiration reservoir. This integrated configuration supports a workflow in which the device 100 is positioned at a skin site, the blade is advanced and retracted via the actuator interface to form an incision, and aspiration is performed via the syringe while the device 100 remains at the skin site. Additionally, the upright orientation depicts external accessibility of actuator interface 120 and syringe plunger 141 while internal components remain within the housing formed by body 101. Additionally, FIG. 5A provides structural support for embodiments wherein blade deployment and retraction establishes a fluid connection between a foot cavity 136 of device 100 and a subcutaneous fluid collection 151 so that wound fluid is aspirated into the syringe through an internal flow path of device 100.

FIG. 5B is a perspective transparent view of an alternative embodiment of an incision and drainage device in a tilted orientation. FIG. 5B shows incision and drainage device 100 corresponding to the device shown in FIG. 5A, with the assembly presented in a tilted orientation wherein body long axis A_{B} is positioned nonorthogonal to foot long axis A_{F}. The transparent rendering depicts an integrated handheld instrument wherein a negative-pressure source is carried within an upper region of the assembly and a tissue-facing interface is carried at a lower region, while internal structures remain visible through the housing. Additionally, the tilted presentation conveys that incision and aspiration functions may be performed while device 100 is held at a non-vertical hand position, such that a user can maintain contact between the lower region of device 100 and a treatment site while accessing controls and aspiration components at the upper region.

In one aspect, the tilted orientation of device 100 depicts an arrangement in which an internal blade actuation assembly 161 and a syringe-supported aspiration volume 143 are aligned along an inclined axis within the housing. Additionally, the transparent view shows that an actuator interface on a side of device 100 remains exposed and reachable in the tilted configuration, enabling advancement and retraction of blade 121 along an internal guide path while device 100 remains supported on tissue. The illustrated geometry indicates that the axial travel direction of the blade and a stroke direction of the aspiration plunger may be coordinated with the inclined axis A_{B} so that a user can execute an incision-and-retraction sequence followed by aspiration without changing the grasp on device 100.

FIG. 5B additionally shows that a fluid communication pathway extends within device 100 between a drainage opening 106 of septum 103 at the tissue-facing interface and a proximal interface that communicates with the aspiration volume, as in some embodiments. The transparent depiction shows that the internal cavity and associated passages are routed through body 101 in the tilted configuration so that negative pressure generated at the syringe 140 (or other vacuum source) is transmitted to drainage opening 106 when a fluid path is established following blade actuation. Furthermore, the lower region at foot 104 of device 100 is shown as a widened base configured to support placement against skin and to assist with maintaining a seal condition adjacent an incision site during aspiration while the upper region remains inclined.

FIG. 5B additionally depicts device 100 in a configuration that supports procedural access near adjacent anatomy by positioning the aspiration components at an angle relative to the tissue-facing interface. The tilted orientation indicates that a coupled syringe 140 can be positioned with lateral clearance relative to the skin surface while negative pressure is applied, which may reduce interference between the syringe and surrounding tissue contours during aspiration. Furthermore, the view of FIG. 5B is consistent with device 100 being held at an inclined angle while blade 121 is deployed and returned to a shielded condition within body 101, after which aspirated subcutaneous fluid is directed through the internal pathway into aspiration volume 143 for containment.

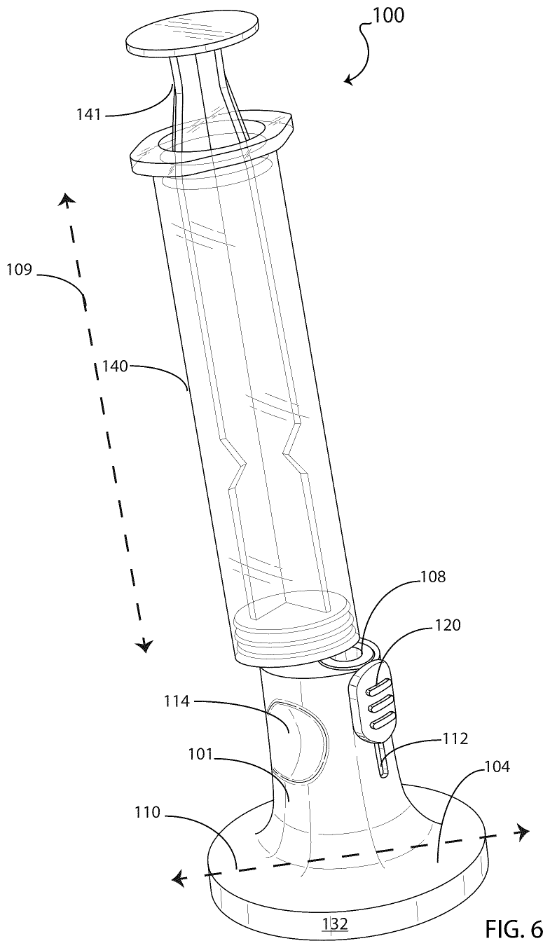

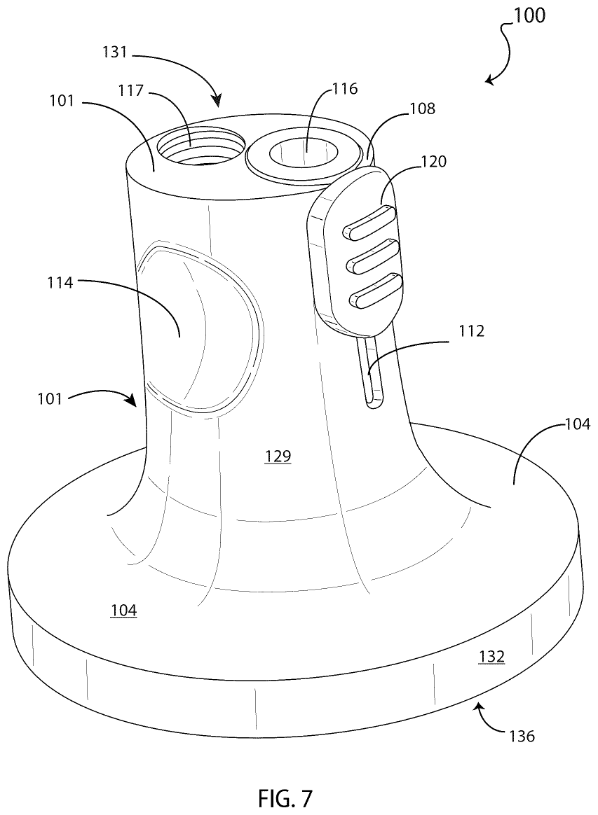

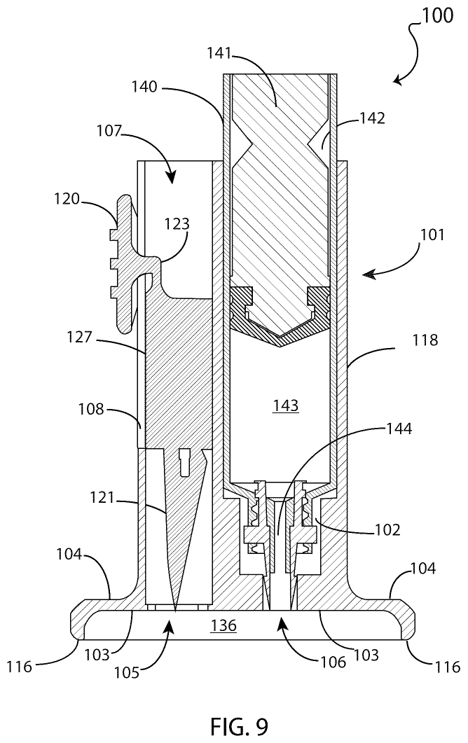

FIG. 6 is a perspective view of an alternative embodiment of an incision and drainage device including a body supporting a syringe and an actuator configured to advance and retract a blade. FIG. 6 shows device 100 wherein syringe 140 is carried by a body 101 to form a handheld assembly configured for sequential incision formation and aspiration. Syringe 140 includes plunger 141 that extends from a proximal end of the syringe 140 for user actuation while the body 101 is supported at a distal region on tissue. Body 101 presents a widened base region, foot 104, that provides a tissue-facing interface, while an exterior actuator interface 120 is disposed on an external side surface of body 101 for controlling an internal blade assembly described elsewhere in the present disclosure. The arrangement shown in FIG. 6 additionally supports a workflow wherein a user maintains the body 101 positioned relative to a treatment site while alternating between actuation of actuator interface 120 and actuation of plunger 141. In some embodiments, including that shown in FIG. 6, body 101 lacks a syringe receiver, rather, syringe 140 couples to body 101 at a syringe mount 102 (shown, e.g., by FIGS. 7 and 9 and described below).

In some embodiments, body 101 includes a distal base region having a rim 116 configured to contact skin surface 150 to support alignment of device 100 relative to a subcutaneous fluid collection targeted for drainage and aspiration. Foot 104 at the base region transitions to an intermediate region of body 101 that provides external surfaces for manual stabilization during actuation. In some embodiments, for example, body 101 includes a grip feature 114 formed as a lateral recess to receive a user's finger and resist rotation of body 101 while actuator interface 120 is manipulated. In some embodiments, the distal region of the body 101 encloses a septum 103 and a foot 104 coupled to septum 103, where in septum 103 includes a blade opening 105 and a drainage opening 106 (not shown in FIG. 6) that cooperate with an internal blade 121 and an internal fluid path of body 101.

In some embodiments, syringe 140 is coupled to the body 101 at a syringe mount 102 disposed at septum 103 of body 101. Syringe mount 102 is configured to retain syringe 140 in a fixed spatial relationship to foot 104 at the distal base region during use. Additionally, syringe mount 102 provides a flow passage between the drainage opening of the septum 103 and a distal outlet region of syringe 140 such that withdrawal of plunger 141 transmits negative pressure through syringe mount 102 of septum 102 through body 101 toward the tissue-facing interface. In some embodiments, syringe 140 is removably coupled to syringe mount 102 enabling exchange of the syringe 140 as a receptacle while retaining the body 101 and the actuator interface 120 as a single assembly positioned for continued use. In some alternative embodiments, syringe 130 is non-removably coupled to septum 103 at syringe mount 102, wherein syringe 140 cannot be removed for emptying or exchanged while aspirating fluid collection 151.

In some embodiments, actuator interface 120 is disposed on an exterior region of body 101 and is arranged for translation relative to body 101 to advance and retract a blade 121 housed within inner volume 107. Actuator interface 120 is configured to transmit user input to an internal slide mechanism that moves along a guided path within the body 101, and the guided path may be established by a slot and tab interface as described with respect to FIGS. 3-5, in some embodiments. Additionally, movement of the actuator interface 120 advances the blade 121 through blade opening 150 of septum 103 to create an access path at the treatment site, and subsequent movement of actuator interface 120, in some embodiments, retracts blade 121 back into a shielded position within the body 101. Retraction of blade 121 after incision formation supports subsequent aspiration through drainage opening 106 of septum 103 while increasing safety associated with use of device 100 by reducing the risk of injury to the patient and the user from external exposure of blade 121.

In one aspect, FIG. 6 depicts the device 100 with body long axis A_{B} aligned with a longitudinal direction of syringe 140 with a distal tissue-facing interface at foot 104 arranged at an offset angle relative to a central axis (not shown) of body 101. Septum 103 (not shown in FIG. 6) may define a septum long axis 110 that is orthogonal to the body long axis 109 such that the syringe 140 is laterally offset from foot 103 of distal base region when rim 116, in some embodiments, contacts tissue. This geometry as shown in FIG. 6 provides clearance for user hand placement around body 101 and access to actuator interface 120 and plunger 141 while maintaining contact between foot 103 and the treatment site during incision formation and aspiration.

FIG. 7 is a perspective view of an alternative embodiment of a device body lacking an elongated syringe received and including a base configured to contact skin, a grip recess, an actuator lobe with a longitudinal slot, and an upper interface forming a septum configured to couple to a syringe. FIG. 7 shows an alternative embodiment of a body 101 for an incision and drainage device 100. Body 101 defines a proximal interface feature 117 configured to couple with a vacuum source, such as a syringe, and a distal interface feature 117 configured to contact tissue during an incision and aspiration sequence as described with respect to FIGS. 1-6. Body 101 provides structural support for an internal blade path and an internal fluid pathway within an inner volume and further supports external access to an actuator interface 120 for advancing and retracting a tissue puncture means 134 while distal interface region 132 remains positioned at a treatment site. Tissue puncture means 134 is a blade 121, such as a scalpel blade, in some embodiments. In some embodiments, skin puncture means is a needle which may be hollow or solid.

In some embodiments, body 101 includes an enlarged foot 104 that defines a rim 116 disposed around a perimeter of foot 104 and transitions into an upwardly extending neck portion 129. The base region includes rim 116 disposed at a distal interface region 132 of body 101 and arranged to contact skin surface 150 during use, with rim 116 cooperating with adjacent wall geometry of foot 104 to define a tissue-facing footprint that supports stable placement during blade actuation and aspiration. Neck portion 129 extends away from foot 104 along body long axis 109 thereby positioning proximal interface region 131 away from the tissue-facing footprint to provide clearance for user hand placement and for coupling of the vacuum source.

In some embodiments, proximal interface region 131 of foot 104 includes a syringe mount 102 configured to mechanically retain a syringe 140 and to place an outlet region of the syringe 140 in fluid communication with the inner volume 107 of the body 101. Additionally, the proximal interface region includes drainage opening 106 adjacent the syringe mount 102, where drainage opening 106 is configured to provide access for assembly of internal components, routing of internal structures, or coupling of an accessory component associated with incision and drainage device 100. Furthermore, the proximal interface region supports embodiments wherein syringe 140 is removably mounted to syringe mount 102 for replacement after aspiration while the body 101 remains positioned at the tissue-facing footprint-tissue interface for continued use with a subsequent syringe 140.

In some embodiments, body 101 includes grip feature 114 disposed on a lateral side surface of neck portion 129. Grip feature 114 is illustrated as a recessed region configured to receive a user finger and to provide a friction surface during manipulation of the actuator interface 120. Additionally, grip feature 114 is positioned between proximal interface region 131 and rim 116 so that a user hand can stabilize body 101 while maintaining access to proximal interface region 131 and while maintaining contact between rim 116 and skin surface 151 or other tissue at distal interface region 132.

In some embodiments, actuator interface 120 is disposed on a side region of body 101 and includes a slot 112 extending along a direction parallel to the body long axis 109. Slot 112 is configured to guide translation of an actuator member, such as tab 123 coupled to a button portion of the actuator interface 120, to advance and retract blade 121 along an internal guide path within the inner volume 107. In some embodiments, actuator interface 120 is positioned adjacent grip feature 114 so that a user can oppose actuation forces using the grip feature 114 while translating the actuator interface 120 along the slot 112, thereby supporting controlled blade deployment through blade opening 105 of septum 103 and subsequent blade withdrawal to a shielded position within actuator lobe 108 of body 101. Furthermore, the relative arrangement of actuator interface 120, slot 112, and proximal interface region 131 supports embodiments wherein aspiration through drainage opening 106 is performed after blade retraction while rim 116 remains engaged with tissue.

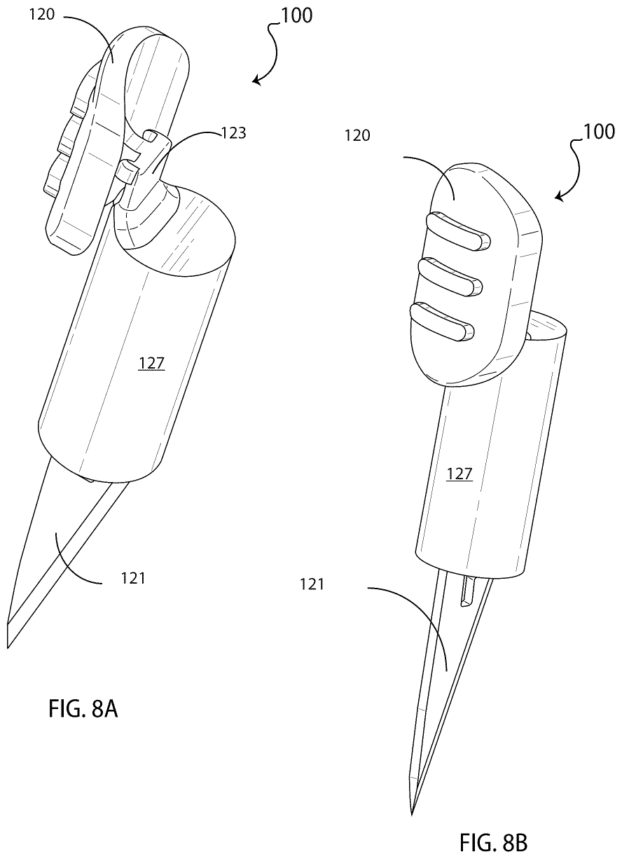

FIG. 8A is a perspective view of an actuator coupled to a blade of an incision and drainage device. FIG. 8B is an alternative perspective view of the actuator coupled to the blade of an incision and drainage device. As in some embodiments, FIG. 8A shows a portion of an incision and drainage device in which actuator interface 120 is mechanically coupled to tissue puncture means 134 by tab 123. As shown in FIGS. 8A-B, tissue puncture means 134 is a scalpel blade, in some embodiments, This is not, however, meant to be limiting. Tissue puncture means 134 may be a scalpel blade such as a #11 blade (as shown in the several drawing figures), #10 blade, #15 blade, etc.; a hollow needle such as a #14G, #16G, #18G, #20 G, or a #22Ghollow needle, etc.; or a solid needle such as a #14G, #16G, #18G, #20 G, or a #22G solid needle, etc. In some embodiments, tissue puncture means 134 is a hollow or a solid needle larger than a #14 needle. In some embodiments, tissue puncture means 134 is a hollow or a solid needle smaller than a #20G needle.

In the illustrated perspective, actuator interface 120 provides an external user interface for applying an actuation force. Tissue puncture means 134 extends distally relative to actuator tab 123 to effect a blade-deployed condition used during an incision event. Additionally, the coupling at tab 123 within slot 112 (shown in FIGS. 1 and 3-7, e.g.) between actuator interface 120 and tissue puncture means 134 supports translation of tissue puncture means 134 along a controlled path during use.

In some embodiments, tissue puncture means 134 is an elongated cutting member having a distal tip and one or more sharpened edges configured to form an incision at a treatment site when advanced from a housing of the device. Additionally, tissue puncture means 134 includes a proximal region that is retained by an internal carrier, such as slide 122 as shown in FIGS. 8A-B and the several drawing figures, so that puncture means 134 moves in response to displacement of actuator tab 120 rather than being independently handled by the user. The depicted extended position indicates that blade 121 is advanced to a stroke endpoint that places the cutting portion beyond a surrounding protective structure, such as blade opening 105 or similar opening of body 101, for example, while the proximal region remains coupled to the actuation mechanism.

In some embodiments, actuator tab 120 includes an enlarged contact surface having contours that support engagement by a user finger during translation along a body-mounted guide feature described elsewhere in the present disclosure. Additionally, actuator interface 120 includes a tab 123 linkage extending from the contact surface of interface 120 toward the blade-carrying portion of the assembly so that motion of the interface 120 is transmitted to puncture means 134. In some embodiments, actuator tab 123 is oriented to move along a direction that corresponds to the deployment direction of puncture means 134, thereby supporting linear advancement of cutting means 134 to the extended position and subsequent return of cutting means 134 to a shielded position when the actuator interface 120 is displaced in an opposing direction.

In some embodiments, the mechanical coupling between actuator interface 120 and puncture means 134 establishes a single actuation interface for both blade/needle advancement and blade/needle retraction. Additionally, positioning actuator interface 120 adjacent of puncture means 134 of the actuator assembly enables a compact linkage in which the user displacement of actuator interface 120 produces a corresponding change in blade/needle extension and/or retraction. Maintaining an extended position of puncture means 134 provides structural support for embodiments wherein user actuation is performed while the device body remains positioned at a skin surface, wherein puncture means 134 may be advanced for piercing and entry of subcutaneous fluid collection 151 and thereafter retracted by reversing movement applied to actuator interface 120.

As in some embodiments, FIG. 8B shows actuator interface 120 mechanically coupled to blade 121 through tab 123 and slide 122. Actuator interface 120 provides a user-contact surface that is displaced to control translation of blade 121 between a blade-deployed condition and a retracted condition with respect to blade opening 105 within septum 103 of body 101. Additionally, blade 121 is coupled to actuator tab 123 such that movement applied to actuator tab 123 via actuator interface 120 is transmitted into a corresponding change in blade extension relative to a surrounding support structure of the actuator assembly.

In some embodiments, the retracted blade or other puncture means position corresponds to a blade-shielding configuration wherein a cutting portion of blade 121 is drawn proximally relative to the deployed position to reduce exposure outside body 101 of device 100 during handling and aspiration. Additionally, blade 121 remains mechanically retained by a blade mount 127 of the actuator assembly so that blade 121 is not independently handled by a user during transitions between the deployed and retracted positions. Maintaining blade 121 in the retracted position supports use cases in which the device is positioned at a treatment site after an incision event, while a negative-pressure source is actuated to aspirate wound fluid without an exposed cutting edge at the tissue-facing interface.

In some embodiments, actuator interface 120 includes a contoured outer surface configured to be engaged by a user finger and translated along a guided path provided by a device body as described elsewhere in the present disclosure. Additionally, actuator interface 120 is arranged to move along an axis corresponding to a blade translation direction so that a single manual input both advances blade 121 for incision formation and retracts blade 121 to the retracted configuration fully within body 101 of device 100. The cooperative coupling between actuator interface 120 and blade 121 supports repeatable stroke endpoints that correspond to an extended blade configuration used to traverse blade opening 105 of septum 103 and a retracted blade configuration wherein blade 121 does not traverse blade opening 105.

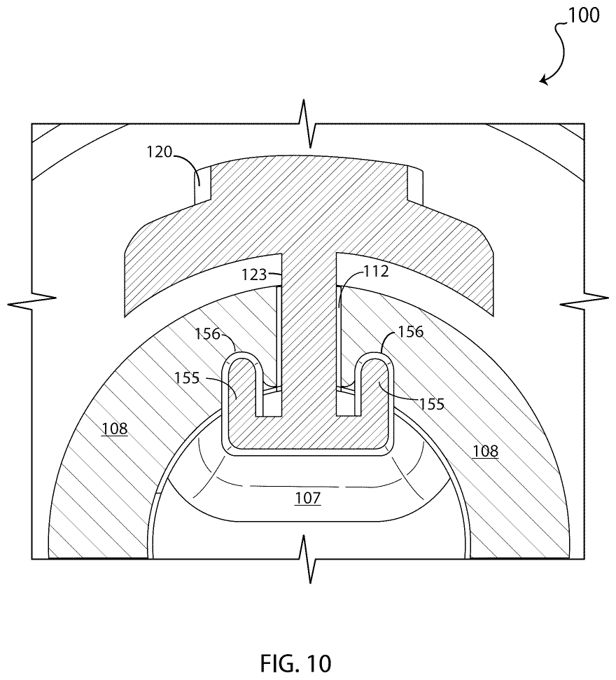

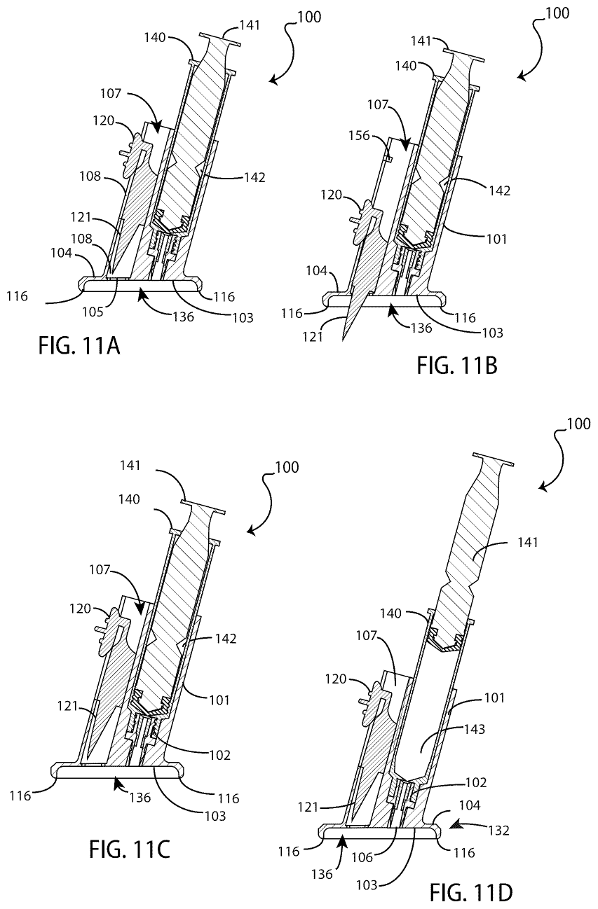

In some embodiments, the retracted position is maintained by interaction between actuator tab 120 and a retention feature of the device body, such as a detent described elsewhere in the present disclosure, (See FIGS. 10-11D) to resist unintentional advancement of blade 121. Additionally, retaining actuator interface 120 in the retracted position supports device workflows wherein blade 121 is returned to a shielded state before aspiration is initiated and remains shielded during aspiration and during removal of device 100 from a patient. FIG. 8B provides structural support for claim limitations reciting an actuator configured to advance and retract a blade, with actuator interface 120 providing an external interface and blade 121 providing a cutting member translated by additional elements of the actuator mechanism.

FIG. 9 is a partial sectional view of an incision and drainage device illustrating an internal blade actuation assembly and a fluid path between a foot cavity and a syringe interface. As in some embodiments, FIG. 9 shows incision and drainage device 100 having an internal arrangement that couples blade actuation to a tissue-facing region of body 101 while also coupling aspiration to syringe 140. The sectional view depicts syringe 140 carried at an upper region of body 101 via syringe mount 102, with plunger 141 positioned for user manipulation to create negative pressure within syringe 140. Furthermore, the illustrated section shows inner volume 107 within body 101 providing space for both a blade translation path and a fluid communication pathway that places a distal region of device 100 in fluid communication with syringe 140.

In some embodiments, syringe mount 102 retains syringe 140 such that an outlet region of syringe 140 is coupled in fluid communication with drainage opening 106 of septum 103. The coupling between syringe 140 and body 101 provides a flow interface that communicates negative pressure from a syringe aspiration volume 143 to a passage within body 101, such as drainage opening 106 for example, without exposure of aspirated material to an exterior of device 100. Thus, withdrawal of plunger 141 increases volume within aspiration volume 143 and communicates negative pressure through drainage opening 106 toward a distal fluid inlet region described below.

In some embodiments, the distal region of body 101 includes septum 103 disposed adjacent a tissue-facing end of device 100, where septum 103 is traversed by at least two openings, namely, blade opening 105 and drainage opening 106, in some embodiments. Foot 104 extends from septum 103 and defines a foot cavity 136 positioned to overlie a treatment site when rim 116 contacts a skin surface. In some embodiments, foot cavity 136 is a shallow cavity having minimal depth which completely fills with tissue whereon device 100 is positioned at the treatment site. This shallow configuration is advantageous, at least in that (1) skin at and surrounding the treatment site is protected from contamination with potentially infections fluid, blood, or other material flowing from fluid collection 151 into aspiration volume 143 of device 100; and (2) skin and adjacent tissue filing foot cavity 135 distribute a sealing force which maintains a vacuum within device 100 when performing an incision and drainage procedure, e.g., distributing a negative pressure/vacuum force across a larger area thus minimizing negative pressure per unit area applied to the skin and underlying tissue. Drainage opening 106 of septum 103 is positioned to place foot cavity 136 in fluid communication with aspiration volume 143, while blade opening 105 provides a guided passage through which blade 121 traverses during an incision event.

As FIG. 9 further illustrates, in some embodiments an actuator arrangement wherein actuator interface 120 is coupled to blade 121 through an internal translation member positioned within actuator lobe 108. In some embodiments, the internal translation member is tab 123. Actuator interface 120 includes a button portion disposed outside an actuator lobe 108 formed by body 101 and coupled to blade mount 127 by tab 123, with tab 123 extending through slot 112 to transmit user motion of interface 120 into additional components of the actuator mechanism contained within actuator lobe 108. Blade mount 127 is mechanically coupled to blade 121 via blade mount 127 such that displacement of actuator interface 120 translates blade mount 127 and produces corresponding advancement and retraction of blade 121 relative to septum 103.

In some embodiments, the partial sectional view of FIG. 9 supports a coordinated sequence in which blade 121 is advanced through blade opening 105 to form an incision while device 100 is positioned with rim 116 contacting tissue, and blade 121 is then retracted through blade opening 105 to a shielded position within actuator lobe 108. After blade 121 is retracted, drainage opening 106 provides a fluid inlet from the foot cavity to aspiration volume 143 so that negative pressure generated by manipulation of plunger 141 draws wound fluid through drainage opening 106 and into aspiration volume 143. Furthermore, the arrangement shown in FIG. 9 enables actuation and aspiration to be performed while body 101 remains positioned at the treatment site, with blade motion guided by septum 103 and aspiration communicated through inner drainage opening 106 into aspiration volume 143 of syringe 140, in some embodiments.

FIG. 10 is an enlarged sectional view through an actuator lobe. FIG. 10 shows actuator tab 123 disposed through slot 112. The enlarged section depicts a retention interface used to hold an actuator assembly in a retracted configuration during handling and during aspiration, with actuator tab 123 extending through a wall region of actuator lobe 108. In some embodiments, tab 123 additionally comprises a surface feature 133 configured to be received by a detent 156. The sectional geometry illustrates how detent 156 provides a mechanical stop that limits travel of actuator tab 123 along a direction of actuation associated with blade translation described with respect to FIGS. 8A-9.

In some embodiments, detent 156 is formed as a recessed pocket or notch in actuator lobe 108 that presents opposing detent surfaces positioned to engage corresponding latch surfaces of actuator tab 123. The illustrated engagement places a widened portion of actuator tab 123 within detent 156 such that actuator tab 123 is resisted from longitudinal displacement relative to actuator lobe 108 without an unlocking action by a user. Actuator lobe 108 may define a slot 112 through which actuator tab 123 projects, wherein detent 156 may be positioned along slot 112 at a location corresponding to a retracted endpoint of travel of actuator tab 123. The detent engagement provides tactile feedback indicating that actuator tab 123 has reached the retained position.

In some embodiments, the retention interface shown in FIG. 10 is implemented with deformation and recovery of one or more polymeric features to permit selective engagement and disengagement. Actuator tab 123 can include a resilient latch portion that elastically deflects during entry into detent 156 and returns toward an undeformed state when seated, thereby retaining actuator tab 123 against reverse movement. Additionally, actuator lobe 108 can include opposing resilient arms or a narrowed throat region that temporarily deflects as actuator tab 123 is pressed into detent 156 and then returns to capture the tab. Furthermore, an unlocking action can include applying a force to actuator tab 123 that produces lateral deflection relative to actuator lobe 108 and clears the latch surfaces from detent 156, after which actuator tab 123 is translated along slot 112.

In some embodiments, seating surface feature 155 of actuator tab 123 in detent 156 corresponds to a blade-shielding configuration wherein an associated blade mount 127 and blade 121 are positioned such that blade 121 does not traverse blade opening 105 of septum 103. Additionally, detent 156 limits unintended advancement of blade 121 when the device is manipulated to actuate syringe 140 for aspiration through drainage opening 106, thereby reducing exposure to a cutting edge at a tissue-facing region of the device. FIG. 10 provides structural support for implementations in which actuator lobe 108 comprises detent 156 configured to retain actuator tab 123 at a retracted endpoint and to be releasable by user input to permit subsequent blade advancement during an incision event.

FIG. 11A is a sectional view of the incision and drainage device in a retracted configuration with a blade positioned within an actuator lobe prior to incision. FIG. 11B is a sectional view of the incision and drainage device in an incising configuration with a blade advanced through a septum and outward from a foot. FIG. 11C is a sectional view of the incision and drainage device in a post-incision configuration with the blade retracted to a shielded position within the actuator lobe. FIG. 11D is a sectional view of the incision and drainage device during aspiration with a syringe plunger withdrawn.

As in some embodiments of device 100, FIG. 11A shows incision and drainage device 100 in a pre-incision configuration wherein blade 121 is in a retracted position within a tissue-facing portion of body 101. In the illustrated section, syringe 140 is mounted to body 101 and includes plunger 141 positioned for user manipulation to generate negative pressure within syringe inner volume 142. A distal region of body 101 includes rim 116 positioned to define a skin-contact interface and to surround a foot 104 that cooperates with septum 103 to define a foot cavity 136 adjacent the treatment site. Furthermore, the sectional depiction shows inner volume 107 extending within body 101 to provide space for a blade translation path.

In some embodiments, the retracted configuration of FIG. 11A positions blade 121 fully within inner volume 107 proximal to septum 103 such that blade 121 does not traverse blade opening 105. Additionally, blade 121 is mechanically coupled to actuator interface 120 through an internal translation assembly disposed within actuator lobe 108, including blade mount 127 coupled to a user-accessible member by tab 123 that extends through slot 112. Furthermore, the stroke endpoint shown in FIG. 11A corresponds to a blade-shielding arrangement wherein a cutting portion of blade 121 is retained within the distal portion of device 100 while rim 116 remains available to contact a skin surface.

In some embodiments, septum 103 further includes drainage opening 106 positioned to place foot cavity 136 in fluid communication with inner volume 107 when syringe 140 is actuated to create aspiration volume 143 (shown in FIG. 11D, e.g.). Maintaining blade 121 in the retracted position reduces exposure of a cutting edge at the tissue-facing end of device 100 while plunger 141 is manipulated to communicate negative pressure through a fluid communication path of body 101. The sectional views for FIGS. 11A-D illustrate that the aspiration pathway from the distal region to syringe 140 remains enclosed within body 101 so that aspirated fluid can be directed toward syringe inner volume 142 forming aspirated volume 143 after an access path to subcutaneous fluid collection 151 is created in a subsequent blade-advanced configuration.

As in some embodiments, FIG. 11A also depicts a body geometry wherein a longitudinal axis of syringe 140 is angled (i.e., nonorthogonal) relative to the tissue-facing interface at rim 116 of foot 104, such that the user can access plunger 141 while maintaining placement of rim 116 at the skin site. Such an angled arrangement positions actuator lobe 108 and slot 112 along an exterior side of body 101 to support guided translation of actuator interface 120 between the retracted configuration shown and an advanced configuration used for incision formation. The configuration of FIG. 11A also supports workflows wherein blade 121 is retained in a retracted state during device positioning, during aspiration, and during removal of device 100 from the skin site. Fe

According to an embodiment, FIG. 11B is a sectional view of incision and drainage device 100 in an incising configuration wherein blade 121 is advanced distally relative to body 101 and traverses blade opening 105 to extend beyond a tissue-facing region of the device 100. The section illustrates syringe 140 mounted to body 101 and aligned for user manipulation of plunger 141, while the distal region of body 101 is configured to be positioned at a skin site during blade deployment. Rim 116 is shown at distal interface region 132 of body 101 and defines a perimeter that can contact the skin surface while blade 121 is translated to create an incision. FIG. 11B also depicts blade 121 advanced from a shielded position within foot 104 to a deployed position in which a cutting portion of blade 121 is exposed outside device 100 for tissue entry.

In some embodiments, the distal region of body 101 includes septum 103 coupled to foot 104, with septum 103 defining blade opening 105 and drainage opening 106. Blade opening 105 provides a guided passage through septum 103 such that blade 121 traverses blade opening 105 when device 100 is in the incising configuration shown in FIG. 11B. Foot 104 defines foot cavity 136 laterally defined by rim 116, with foot cavity 136 arranged to overlie the treatment site while device 100 is held against tissue. Deployment of blade 121 through blade opening 105 positions blade 121 to pierce the skin and enter subcutaneous tissue to establish an access path that is used for subsequent drainage through foot cavity 136.

In some embodiments, advancement of blade 121 shown in FIG. 11B is produced by translation of actuator interface 120 along actuator lobe 108, with user motion transmitted through tab 123 extending through slot 112. The sectional view depicts an internal translation assembly in which blade mount 127 is coupled to blade 121 via blade mount 196 such that longitudinal displacement of blade mount 127 advances blade 121 through septum 103. Additionally, the guided relationship between tab 123 and slot 112 constrains actuator motion relative to body 101 to control a stroke that corresponds to a deployed endpoint shown in FIG. 11B. Additionally, the internal coupling between actuator interface 120, blade mount 127, and blade 121 supports a configuration wherein blade deployment occurs with rim 116 remains positioned at the skin site.

As in some embodiments, FIG. 11B further illustrates that syringe 140 remains coupled to body 101 during the incising step so that aspiration can be performed after blade 121 is retracted. Syringe inner volume 142 is shown within syringe 140, and plunger 141 is positioned to be withdrawn (creating aspiration volume 143) after the incision is formed and blade 121 is returned to a shielded position within foot 104. Syringe inner volume 142 is a space largely occupied by plunger 141 prior to withdrawal of plunger 141 to form aspiration volume 143, as shown in the several drawing figures discussed herein above.

Additionally, drainage opening 106 remains available to place the foot cavity in fluid communication with inner volume 107 of body 101 when the blade is retracted from blade opening 105. FIG. 11B also depicts a coordinated arrangement wherein blade 121 provides tissue access through blade opening 105 of septum 103 while the aspiration pathway through body 101 and syringe 140 remains enclosed for collection of wound fluid in a subsequent aspiration step.

As in some embodiments, FIG. 11C is a sectional view of incision and drainage device 100 in a post-incision configuration wherein blade 121 is retracted proximally relative to the incising configuration of FIG. 11B. Syringe 140 remains coupled to body 101, with plunger 141 positioned for subsequent user manipulation to generate negative pressure within aspiration volume 143. A distal interface region 132 of body 101 includes rim 116 arranged to define a tissue-contact interface surrounding foot 104, with septum 103 disposed proximal to foot 104 within the distal interface region 132.

In some embodiments, blade 121 is positioned within foot 104 such that blade 121 does not traverse blade opening 105 of septum 103. Retraction of blade 121 is produced by an actuator transmission within actuator lobe 108, wherein actuator interface 120 drives blade mount 127 through tab 123 extending through slot 112, and blade mount 127 is coupled to blade 121. The retracted stroke endpoint places a cutting portion of blade 121 wholly within a distal portion of device 100 to reduce exposure at the tissue-facing end while device 100 remains positioned for aspiration.

As in some embodiments, FIG. 11C further depicts a fluid communication arrangement wherein drainage opening 106 of septum 103 places a cavity 136 of foot 104 in fluid communication with inner volume 107 of body 101 while blade 121 is retracted. Additionally, cavity 136 provides drainage opening 106 passage that communicates with syringe mount 102 and syringe 140 such that negative pressure generated by displacement of plunger 141 is communicated toward drainage opening 106. Furthermore, the configuration of FIG. 11C supports a sequence in which an access path formed by the preceding incision remains available at the treatment site while aspiration is initiated through drainage opening 106 with blade 121 retained within inner volume 107.

In some embodiments, the actuator position associated with FIG. 11C is maintained by a retention interface between an actuator tab surface feature 155 and a detent 156 (as shown in FIG. 11B) of actuator lobe 108, as described with respect to FIG. 10, to resist unintended advancement of blade 121 during aspiration and device handling. Maintaining the actuator interface 120 at the retracted endpoint retains blade mount 127 and blade 121 in a corresponding retracted position while syringe 140 is actuated to draw fluid from the subcutaneous fluid collection 151 through drainage opening 106 into aspiration volume 143. FIG. 11C also illustrates a condition wherein incision formation and blade withdrawal are completed prior to aspiration, with the aspiration flow path remaining enclosed within body 101 between the distal region and syringe aspiration volume 143.

As in some embodiments, FIG. 11D is a sectional view of incision and drainage device 100 during an aspiration operation in which plunger 141 of syringe 140 is withdrawn relative to body 101. Syringe 140 is coupled to body 101 at syringe mount 102 and defines syringe inner volume 142 that converts to aspiration volume 143 as plunger 141 is displaced, thereby establishing negative pressure for drawing fluid and material from the subcutaneous fluid collection 151 through device 100. Note that body 101 is positioned with rim 116 at a tissue-facing end region and with foot 104 disposed within the perimeter of rim 116 to define a cavity 136 overlying an incision site created in a preceding step.

In some embodiments, FIG. 11D illustrates that blade 121 remains in a retracted configuration during aspiration such that blade 121 does not traverse blade opening 105 of septum 103. Blade 121 is mechanically coupled to actuator interface 120 through blade mount 127 disposed within actuator lobe 108, with tab 123 extending through slot 112 to transmit user motion that previously advanced and retracted blade 121. Actuator interface 120 is retained at a retracted endpoint during the aspiration state, including via engagement between surface feature 155 and detent 156 as described with respect to FIG. 10, to resist translation of blade mount 127 that would otherwise advance blade 121.

As in some embodiments, FIG. 11D depicts a fluid communication path established through septum 103 and body 101 while blade 121 is retracted. As noted, septum 103 includes drainage opening 106 positioned to couple the cavity of foot 104 in fluid communication with inner volume 107 of body 101, and aspiration volume 143 through syringe mount 102. Withdrawal of plunger 141 communicates negative pressure from syringe aspiration volume 143 through drainage opening 106 to foot cavity 136 thereby drawing wound fluid from a subcutaneous access path through foot cavity 136 and onward into aspiration volume 143 of syringe 140 for containment.

As will be appreciated from the sectional arrangement shown by FIG. 11D supports aspiration while device 100 remains positioned at the treatment site with rim 116 contacting a skin surface. Maintaining contact between rim 116 and tissue during withdrawal of plunger 14 is desirable to prevent or limit leakage of potentially infectious fluid present at the tissue-facing interface and additionally supports transmission of negative pressure to the incision site through foot 104. Furthermore, the aspiration state shown in FIG. 11D cooperates with the post-incision blade configuration described with respect to FIG. 11C such that the cutting portion of blade 121 remains shielded within the distal region of body 101 while fluid and material from a subcutaneous fluid collection is aspirated through drainage opening 106 into syringe 140.

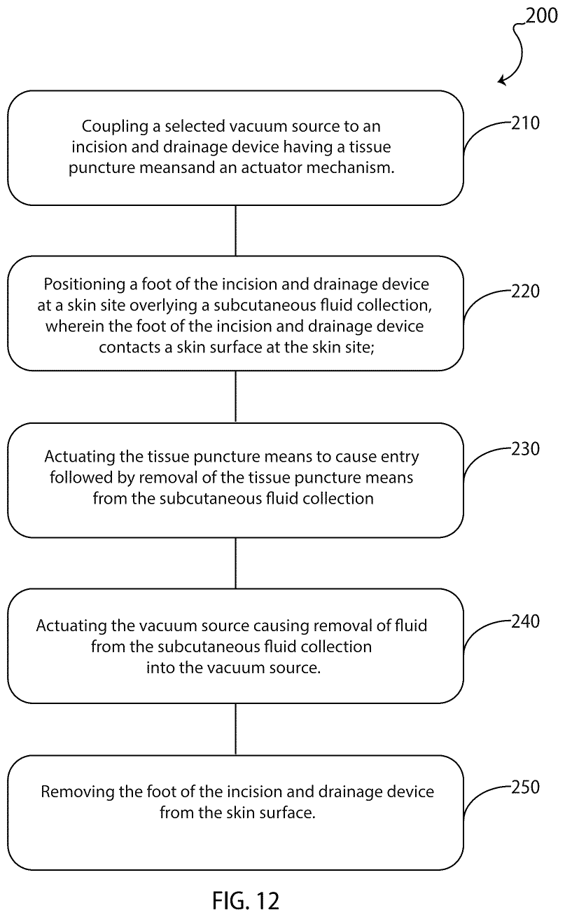

FIG. 12 is a flow chart illustrating a method of using an incision and drainage device. As in some embodiments, FIG. 12 diagrams steps illustrating method 200 for using an incision and drainage device to access and remove fluid from a subcutaneous fluid collection. The flow chart depicts method 200 as a sequence of operation steps including exchanging step 210, incising step 220, retracting step 230, aspirating step 240, and removing step 250. Method 200 is performed using an incision and drainage device having a body that supports a vacuum source, a tissue-facing foot, and an actuator-controlled blade that is advanced through a blade opening and returned to a shielded position to enable aspiration through a drainage opening. Method 200 supports performance of incision formation and aspiration of a subcutaneous fluid collection while the device remains positioned at a skin site.

In some embodiments, exchanging step 210 includes coupling a selected vacuum source to the incision and drainage device prior to tissue access, or replacing the vacuum source during a procedure. In some embodiments, the selected vacuum source is a syringe. In some embodiments, the syringe is disposable. In some embodiments, the selected vacuum source is a length of vacuum tubing terminating in a coupling means, such as a Luer Lok fitting, for example, wherethrough a vacuum is transmitted from a source external to the device, such as “wall suction,” or an external vacuum pump, e.g. Exchanging 210 may include mounting a syringe to a syringe mount of the body, including positioning an outlet region of the syringe in fluid communication with an internal flow path that communicates with a foot cavity. Exchanging step 210 may alternatively comprise decoupling a first syringe after aspiration and coupling a second syringe to continue aspiration or to collect additional material, while either retaining the incision and drainage device body at the treatment site or after temporarily lifting the body from the skin. In some embodiments, exchanging step 210 includes selecting a syringe size based on an expected volume of wound fluid and configuring the device to retain the syringe during subsequent blade actuation.

In some embodiments, incising step 220 comprises positioning a tissue-facing rim of the incision and drainage device on a skin surface adjacent a target site and actuating an actuator to advance a blade through a septum to create a skin incision that reaches a subcutaneous fluid collection. Incising step 220 includes translating the blade along a guided path through an actuator lobe. In some embodiments, associated slot and tab features of device 100 contribute to actuating blade travel along the guided path so that blade travel is constrained along a direction referenced to a body long axis of the device. Incising step 220 additionally comprises advancing the blade to a deployed configuration in which a cutting portion extends beyond the foot to traverse the skin surface. Incising step 220 is performed while maintaining contact between the rim and the skin surface to support alignment of the foot cavity with the target site.

In some embodiments, retracting step 230 comprises reversing actuator motion after completion of incising step 220 to withdraw the blade proximally through the blade opening and into a shielded position within the actuator lobe of the body of the incision and drainage device. Retracting step 230 includes translating a blade mount coupled to the actuator such that the blade is drawn away from the tissue-facing end of the device while the device remains positioned on the skin surface. Retracting step 230 may additionally include, in some embodiments, retaining the actuator in a retracted position using a detent or other retention feature to resist unintended blade advancement during aspiration and handling. Retracting step 230 establishes a post-incision configuration wherein the blade does not traverse the blade opening while a drainage opening remains available to communicate negative pressure to the foot cavity.

In some embodiments, aspirating step 240 comprises generating negative pressure within the coupled vacuum source and communicating the negative pressure through an internal flow path of the body to the foot cavity to draw wound fluid through the drainage opening. In some embodiments utilizing a syringe as the vacuum source, for example, aspirating step 240 comprises withdrawing a syringe plunger to increase syringe inner aspiration volume and thereby aspirate subcutaneous fluid and material into the syringe. Aspirating step 240 is performed following retracting step 230 so that aspiration occurs while the blade remains shielded within the device. Aspirating step 240 includes maintaining the rim against the skin surface to limit leakage and to support vacuum retention within the internal fluid path, urging fluid flow from the subcutaneous fluid collection into the foot cavity and through the drainage opening.

In some embodiments, removing step 250 comprises lifting the incision and drainage device away from the skin surface after completion of aspirating step 240 and maintaining the blade in the retracted position during device handling and disposal. Removing step 250 may additionally include separating the vacuum source from the syringe mount when a removable syringe configuration is used, including maintaining aspirated material within the syringe as a receptacle for containment and transport or disposal consistent with regulations and best practices for handling of biohazard material. Removing step 250 may optionally include, after decoupling the vacuum source, repeating exchanging step 210 to couple a replacement syringe for subsequent procedures or aspiration of additional fluid at the same site. Removing step 250 optimally includes completing the procedure with the blade retained in a shielded state to reduce sharps exposure during post-procedure handling.

Various embodiments of an incision and drainage device have been described herein. The device is configured to allow for controlled entry into a subcutaneous fluid collection, such as an abscess, cyst, hematoma, or the like followed by contained aspiration of contents of the collection into a vacuum source, such as a syringe, vacutainer tube, suction trap, etc. Use of the device, particularly by an inexperienced clinician, streamlines an incision and drainage procedure by integrating incision and aspiration functionality in a single device, providing controlled blade advancement and retraction through an actuator mechanism, and enabling collection of aspirated material in a receptacle for contained disposal. The devices disclosed herein are configured to facilitate more efficient evacuation of fluid from a subcutaneous fluid collection and may reduce handling of exposed sharps and drained material during and after the procedure. Such devices may also decrease hesitancy by a less-experienced clinician performing an incision and drainage procedure.

What is claimed is:

1. An incision and drainage device comprising:
a body having a syringe mount, a septum comprising a blade opening and a drainage opening, a foot extending from the septum bounded by a rim and defining a foot cavity, and an actuator lobe;
a vacuum source coupled to the syringe mount;
an actuator coupled to the actuator lobe; and
a blade mechanically coupled to the actuator interface, wherein the actuator interface is configured to advance and retract the blade;
wherein advancement and retraction of the blade in response to the actuator interface establishes a fluid connection between the foot cavity and a subcutaneous fluid collection in an animal, whereupon a subcutaneous fluid collection may be aspirated through the drainage opening into an aspiration volume fluidly coupled to the syringe mount.

2. The device of claim 1, wherein the body further comprises an elongated syringe receiver.

3. The device of claim 1, wherein the vacuum source is a syringe is removably mounted to the syringe mount.

4. The device of claim 1, wherein the vacuum source comprises a Vacutainer.

5. The device of claim 4. further comprising a closing mechanism configured to contain aspirated material after aspiration, the closing mechanism comprising two semi-circular members configured to close around a diameter of the Vacutainer.

6. The device of claim 1, wherein the blade is manually advanced and is spring-loaded to retract.

7. The device of claim 1, wherein the septum comprises a long axis and the body comprises a long axis, wherein the body long axis is orthogonal to the septum long axis.

8. The device of claim 1, wherein the septum comprises a long axis and the body comprises a long axis, wherein the body long axis is nonorthogonal to the septum long axis.

9. The device of claim 5, wherein the body long axis is about fifteen degrees nonorthogonal to the septum long axis.

10. The device of claim 1, wherein the body comprises a grip feature.

11. The device of claim 1, wherein the rim is disposed opposite the septum and configured for contacting the skin of an animal.

12. The device of claim 1, wherein
the actuator lobe comprises a slot oriented parallel to a long axis of the body;
the actuator comprises a blade mount coupled to an actuator interface by a tab, wherein movement of the tab is constrained by the slot, the tab is positioned with the blade mount inside the actuator lobe in a retracted position prior to an actuation sequence, the actuator interface is positioned outside the actuator lobe of the body, and the tab is disposed through the slot,
wherein the blade mount is configured for movement through the actuator lobe parallel to a long axis of the body in response to movement of the actuator interface transmitted through the tab.

13. The device of claim 9, further comprising a detent in the actuator lobe configured to retain the tab in a retracted position wherein the blade does not traverse the blade opening of the septum.

14. An incision and drainage device comprising:
a body having a syringe mount, a septum comprising a blade opening and a drainage opening, a foot coupled to the septum, and an actuator lobe;
a syringe coupled to the syringe mount;
an actuator coupled to the actuator lobe; and
a blade mechanically coupled to the actuator, wherein the actuator is configured to advance and retract the blade;
wherein advancement and retraction of the blade in response to the actuator establishes a fluid connection between the foot cavity and a subcutaneous fluid collection in an animal, whereupon a wound fluid may be aspirated through the drainage opening into a syringe coupled to the syringe receiver.

15. A method of use for an incision and drainage device to remove fluid from a subcutaneous fluid collection comprising steps:
coupling a selected vacuum source to an incision and drainage device having a tissue puncture means and an actuator mechanism for the tissue puncture means;
positioning a foot of the incision and drainage device at a skin site overlying a subcutaneous fluid collection, wherein the foot of the incision and drainage device contacts a skin surface at the skin site;
actuating the tissue puncture means to cause entry followed by removal of the tissue puncture means from the subcutaneous fluid collection;
actuating the vacuum source causing removal of fluid from the subcutaneous fluid collection into the vacuum source and
removing the foot of the incision and drainage device from the skin surface.

16. The method of claim 15, further comprising a step exchanging the vacuum source.

17. The method of claim 15, wherein the vacuum source comprises a Vacutainer.

18. The method of claim 15, wherein the vacuum source comprises a syringe, and wherein actuating the vacuum source comprises withdrawing a plunger of the syringe.

19. The method of claim 15, further comprising maintaining aspirated material within the vacuum source after removing the incision and drainage device from the skin site.

20. The method of claim 15, wherein the blade is retained in an actuator lobe following blade removal from the subcutaneous fluid collection during actuation of the vacuum source.
